(19) United States
(12) Patent Application Publication
(10) Pub. No.: US 2026/0297137 A1
(43) Pub. Date: Oct. 1, 2026

(54) UNIVERSAL AVIAN FLU VACCINE (AIV)

(71) Applicant: DEI Biopharma, Kampala (UG)

(72) Inventor: Matthias Magoola, Kampala (UG)

(21) Appl. No.: 19/094,937

(22) Filed: Mar. 30, 2025

(51) Int. Cl. C07K 14/005; A61K 39/00

(52) U.S. Cl. CPC C07K 14/005; A61K 2039/55555; A61K 2039/70; C12N 2760/16122; C12N 2760/16134

(57) ABSTRACT

The universal vaccine of the present invention can induce one or more targeted immune responses against a broad range of AIV subtypes by incorporating their antigen-producing sequences for cross-protection against various subtypes of AIV. In another embodiment, the invention is manufactured by recombinant or RNA technology, either for in vitro manufacturing or vivo administration, and administered by parenteral or inhalation route.

### SEQUENCE LISTING

The instant application contains a Sequence Listing, which has been submitted electronically in .xml format and is hereby incorporated by reference in its entirety. Said text file, created on 26 Mar. 2025, is named UNIVERALFLUVACCINE.xml and is 28000 bytes in size.

### FIELD OF THE INVENTION

The universal vaccine of the present invention can induce one or more targeted immune responses against a broad range of AIV subtypes by incorporating their antigen-producing sequences for cross-protection against various subtypes of AIV. In another embodiment, the invention is manufactured by recombinant or RNA technology, either for in vitro manufacturing or in vivo administration, and administered by parenteral or inhalation route.

### BACKGROUND OF THE INVENTION

All avian influenza viruses belong to the genus Alpha influenza virus in the family Orthomyxoviridae and are classified as Influenza A viruses. These viruses are further subtyped based on the two surface glycoproteins hemagglutinin (H1 to H18) and neuraminidase (N1 to N11), resulting in combinations such as H5N1, H5N8, and H7N9. These subtypes vary in pathogenicity and host range, but all originate within this viral genus.

Avian influenza has evolved from primarily a poultry disease to a significant global health security threat. The H5N1 clade 2.3.4.4b has demonstrated unprecedented geographic spread across continents and shows worrisome signs of adaptation to mammals, including recent reports of transmission among dairy cattle in the United States. Since 2021, the epidemic has caused devastating agricultural losses, with over 400 million poultry deaths globally. Although human infections remain limited, they continue to raise concern, primarily occurring in individuals with direct exposure to infected birds. The current vaccine landscape remains inadequate, relying mainly on strain-specific approaches, such as traditional inactivated whole-virus vaccines for poultry and egg-based or cell-based vaccines for pandemic preparedness in humans.

These existing vaccines suffer from critical limitations, including antigenic mismatch due to rapid viral evolution, production delays of 6+ months, insufficient manufacturing capacity for global pandemic response, and restricted protection against specific subtypes only. These shortcomings highlight the urgent need for universal avian influenza vaccine strategies targeting conserved epitopes across multiple subtypes. Up-and-coming are next-generation recombinant protein-based platforms featuring multi-epitope constructs, self-assembling nanoparticles, headless HA constructs, and RNA-based platforms that leverage mRNA technology proven successful during COVID-19.

These approaches target crucial conserved antigens, including the M2e domain, hemagglutinin (HA) stem region, nucleoprotein, matrix protein, and the neuraminidase active site. Despite encouraging advances, significant challenges remain-such as optimizing antigen design, addressing immunodominance, defining correlates of protection, establishing clear regulatory pathways, and ensuring equitable global access. The increasingly concerning avian influenza situation demands urgent, collaborative international efforts to develop, manufacture, and distribute universal vaccine strategies capable of providing broad protection against emerging zoonotic subtypes. Such efforts are essential to transform our approach to pandemic preparedness ultimately.

The economic impact of avian influenza extends well beyond direct poultry losses, affecting entire agricultural supply chains, international trade, and public health systems worldwide. Outbreaks often lead to the mass culling of millions of birds, severe production disruptions, export bans, and heightened market price volatility. These direct effects ripple through related industries, resulting in job losses, undermining food security, and occasionally impacting tourism in severely affected regions. The costs of extensive surveillance programs, treatment of human cases, and the implementation of ongoing prevention and biosecurity measures further compound the financial burden.

Current avian influenza vaccines present a critical yet complex economic consideration. Animal vaccines typically cost between $0.10 and $0.50 per dose, excluding implementation expenses. In comparison, human pandemic-ready vaccines require substantial development investments ranging from $100 to $300 million, with government stockpiling costs estimated at $12 to $25 per dose. Although cost-benefit analyses generally favor preventive measures over emergency responses, optimal strategies vary widely depending on regional conditions, viral strain characteristics, and the capacity of existing infrastructure. Overall, the global annual economic burden of avian influenza—including prevention, outbreak response, and associated financial losses—amounts to billions of dollars, positioning it as a significant concern for agricultural economies and public health systems.

Peptide vaccines are a safer and more economical technology than traditional vaccines. The disadvantage of this technology is its poor immunogenicity. Several experiments have been performed with this vaccine to test its ability to protect birds. The published results showed that the protection reached with the peptide vaccine was lower than 50% in all the challenges tested in the field. In contrast with these results, inactivated virus vaccines (positive control) commonly reach from 90 to 100% of protection. For this reason, the vaccines routinely used as part of eradication programs and in emergencies are based on inactivated viruses. AIV epitopes are well-recognized (Table 1)

TABLE 1
Comprehensive Avian Influenza Virus Epitope: Hemagglutinin (HA) Epitopes

| Epitope Region | Subtype | Sequence | Function/Notes |
|---|---|---|---|
| Site A (140-145) | H5N1 | PQRERRRKK | Highly variable, |
| | | (SEQUENCE NO. 1) | major antibody target |
| Site B (155-165) | H5N1 | LKNNQKIYVQTL | Major neutralizing |
| | | (SEQUENCE NO. 2) | epitope |
| RBD | H5N1 | WLTEKEGSYP | Critical for host cell |
| | | (SEQUENCE NO. 3) | binding |
| HA Stem | H5N1 | GLFGAIAGFIE | Conserved target for |
| | | (SEQUENCE NO. 4) | broadly neutralizing |
| | | | antibodies |
| Antigenic Site Sa | H7N9 | VPNLPFQNAL | Species-specific |
| | | (SEQUENCE NO. 5) | variation |
| Antigenic Site Sb | H7N9 | RTFFLTQGA | Antibody escape |
| | | (SEQUENCE NO. 6) | mutations are |
| | | | common |
| FI6 Binding Site | H1-H16 | Not specified | Universal |
| | | | neutralizing antibody |
| | | | target |
| CR6261 Binding Site | H1, H5 | Not specified | The conserved helical |
| | | | region in HA stem |

Neuraminidase (NA) Epitopes are listed in Table 2.

TABLE 2
Neuraminidase (NA) Epitopes

| Epitope Region | Subtype | Sequence | Function/Notes |
|---|---|---|---|
| Active Site | H5N1 | ILRTQESEC | Conserved enzymatic |
| | | (SEQUENCE NO. 7) | site |
| 150-Loop | H5N1 | IASRSGYSGN | Variable surface |
| | | (SEQUENCE NO. 8) | loop |
| 430-Loop | H5N1 | PCICITPNGSIP | Antigenic domain |
| | | (SEQUENCE NO. 9) | |
| Hemadsorbing | H7N9 | KWVDGTQYRT | Secondary |
| Site | | (SEQUENCE NO. | sialic acid |
| | | 10) | binding site |

Nucleoprotein (NP) Epitopes are shown in Table 3.

TABLE 3
Nucleoprotein (NP) Epitopes

| Epitope | | HLA | | Function/ |
|---|---|---|---|---|
| Region | Subtype | Restriction | Sequence | Notes |
| NP44-52 | H5N1 | HLA-A*0201 | CTELKLSDY | Major CD8+ T |
| | | | (SEQUENCE NO. | cell epitope |
| | | | 11) | |
| NP174-184 | H5N1 | HLA-B*2705 | RRSGAAGAAVK | Conserved |
| | | | (SEQUENCE NO. | across influenza |
| | | | 12) | A |
| NP265-273 | H7N9 | HLA-A*0301 | ILRGSVAHK | Cross-reactive T |
| | | | (SEQUENCE NO. | cell epitope |
| | | | 13) | |
| NP383-391 | Pan- | HLA-B*0801 | ELRSRYWAI | Highly |
| | Influenza | | (SEQUENCE NO. | conserved T cell |
| | | | 14) | epitope |

Matrix Protein (M1) Epitopes are shown in Table 4.

### Matrix Protein (M1) Epitopes

| Epitope Region | Subtype | HLA Restriction | Sequence | Function/Notes |
|---|---|---|---|---|
| M158-66 | Pan- | HLA-A*0201 | GILGFVFTL | Immunodominant, |
| | Influenza | | (SEQUENCE | highly |
| | | | NO. 15) | conserved |
| M1128-135 | H5N1 | HLA-B*2705 | ASCMGLIY | CD8+ T cell |
| | | | (SEQUENCE | epitope |
| | | | NO. 16) | |

M2 Protein Epitopes are shown in Table 5.

### M2 Protein Epitopes

| Epitope Region | Subtype | Sequence | Function/Notes |
|---|---|---|---|
| M2e (2-24) | H5N1 | SLLTEVETPIRNEWGCRCNDSSD | Extracellular |
| | | (SEQUENCE NO. 17) | domain, universal |
| | | | vaccine target |
| M2e (2-24) | H7N9 | SLLTEVETPTRSEWECRCSDSSD | Variant with 5 |
| | | (SEQUENCE NO. 18) | amino acid |
| | | | differences |
| M2e (2-24) | H9N2 | SLLTEVETPTRNGWECKCSDSSD | Common in Asian |
| | | (SEQUENCE NO. 19) | poultry viruses |

Note: Sequences for FI6 and CR6261 binding sites are not specified due to their broad reactivity across multiple HA subtypes.

For designing a multi-epitope polypeptide vaccine against avian influenza, selected epitopes that collectively provide broad protection while immunogenic in the context of a fusion protein. These epitopes are listed in Table 6.

TABLE 6
Selected peptides for avian vaccine

| | |
|---|---|
| M2e sequence | SLLTEVETPIRNEWGCRCNDSSD |
| | (SEQUENCE NO. 20) |
| HA Stem region | GLFGAIAGFIE (SEQUENCE NO. 21) |
| epitope | |
| NP T-cell epitope | ELRSRYWAI (SEQUENCE NO. 22) |
| M1 epitope | GILGFVFTL (SEQUENCE NO. 23) |
| Conserved NA | ILRTQESEC (SEQUENCE NO. 24) |
| active site | |

The suitable linkers include Furin cleavage site RVRRKR (SEQUENCE NO. 25), Furin cleavage site between the hemagglutinin conserved region and other epitopes; rigid helical spacer EAAAKEAAA (SEQUENCE NO. 26), rigid helical linker to maintain spatial separation for the fusion peptide, cathepsin-B cleavable SLLRY (SEQUENCE NO. 27), Cathepsin-B cleavable linker for optimal processing of T-cell epitopes enabling optimal post-translational processing and epitope presentation.

Recombinant Protein Vaccine Construct:
(SEQUENCE NO. 28)
SLLTEVETPIRNEWGCRCNDSSD-RVRRKR-GLFGAIAGFIE-
EAAAKEAAAK-ELRSRYWAI-SLLRY-GILGFVFTL-SLLRY-
ILRTQESEC
RNA Vaccine Construct:
(SEQUENCE NO. 29)
SLLTEVETPIRNEWGCRCNDSSD-GGGGS-GLFGAIAGFIE-
AAY-ELRSRYWAI-GPGPG-GILGFVFTL-GPGPG-
ILRTQESEC

Both constructs are designed to optimize epitope presentation while maintaining the functional integrity of each epitope. The recombinant protein version leverages in vivo processing mechanisms, while the RNA vaccine construct optimizes translation efficiency and proper folding during protein synthesis.

For the first time, the present invention introduces a platform for obtaining a wide range of vaccines that combine polynucleotides encoding viral peptides or recombinant viral peptides containing adjuvants, emulsifiers, molecular adjuvants, and carrier systems. It is proposed that the present invention could develop a universal vaccine to protect birds against one or more viral diseases, particularly the bird flu virus.

### SUMMARY OF THE INVENTION

In one embodiment, the present invention provides a detailed procedure for formulating a universal vaccine that protects against various subtypes of bird flu viruses.

In one embodiment, the formulations are composed of polynucleotides encoding viral peptides, polypeptides, or proteins in different types of plasmids; viral peptides or recombinant viral peptides, along with a carrier or as molecular adjuvant fused to peptides, polypeptides and/or proteins derived from viruses; adjuvants; emulsifiers, molecular adjuvants, and carrier systems.

In one embodiment, the present invention discloses a vaccine formulation capable of inducing cross-protection against different subtypes or subtypes of AIV viruses by (a) polynucleotides encoding peptides, polypeptides, or proteins of the virus; (b) recombinant peptides, polypeptides or proteins of the virus; subtype and (d) adjuvants to enhance the potency of the AIV vaccine.

In one embodiment, the pharmaceutical combination comprising one or more vaccine formulations of the present invention can ensure high protection against AIV by induction of cell-mediated and humoral components of the immunological response.

In one embodiment, the pharmaceutical combination comprising one or more vaccine formulations of the present invention has the advantage of generating immunogenic cross-protection with fewer vaccination campaigns.

In birds, extending the half-life of avian influenza vaccines requires alternative strategies since they lack the neonatal Fc receptor (FcRn), which is essential for albumin- or IgG-based recycling in mammals. One effective approach is using biodegradable nanoparticles or microparticles, such as those made from poly(lactic-co-glycolic acid) (PLGA) or chitosan. These materials enable controlled antigen release and protect vaccine components from enzymatic degradation, thus prolonging their presence in the body. Another promising strategy involves encapsulating antigens in liposomes or immunostimulating complexes (ISCOMs), enhancing antigen stability and facilitating uptake by antigen-presenting cells. Depot-forming adjuvants such as Montanide ISA 70 or ISA 71 are widely used in poultry vaccines to create a local reservoir at the injection site, slowly releasing the antigen over time.

Additionally, fusion of vaccine antigens to avian transferrin or transferrin-binding peptides can leverage transferrin receptor-mediated uptake to enhance immune targeting and antigen retention. Polyethylene glycol (PEG) conjugation, or PEGylation, offers another method to protect peptide antigens from rapid clearance and degradation, although it may sometimes reduce immunogenicity. In contrast, strategies like Fc or albumin fusion, which are effective in mammals, are unsuitable for birds due to the absence of FcRn-mediated recycling. A practical and effective avian flu vaccine formulation might include a peptide or protein antigen encapsulated in chitosan-PLGA nanoparticles, combined with a depot adjuvant like Montanide ISA 70, and delivered via intramuscular or intranasal routes to achieve prolonged antigen exposure and enhanced immune response.

### DETAILED DESCRIPTION OF THE INVENTION

The method described in this patent application illustrates the formulation process to achieve a high-quality vaccine for the AIV.

In one embodiment, the present invention relates to a method to formulate a universal vaccine against one or more subtypes of an AIV.

In one embodiment, the present invention provides immunogenic components to formulate different vaccines to ensure cross-protection against all or different subtypes or subtypes of a virus, such as AIV.

In one embodiment, the present invention provides immunogenic components to formulate vaccines to ensure total or cross-protection against different AIV subtypes combined with one or more immunogenic components. This ensures a high protection that comprises cellular and humoral components of the immunological response.

In one embodiment, the universal vaccine of the present invention can specifically induce one or more targeted immune responses against all or different subtypes of an AIV.

One of the ordinary skills in art is readily recognizing that the present invention can be designed using any combination of polynucleotides derived from AIV.

One of the ordinary skills in art is readily recognizing that the present invention can be designed using a combination of different recombinant peptides, polypeptides, and/or proteins derived from AIV.

Avian influenza viruses (AIVs) present multiple antigenic epitopes essential for immune recognition and vaccine design. The hemagglutinin (HA) protein is the primary target of neutralizing antibodies, with several conserved epitopes in both the head and stalk regions. In H1N1 and H1N2 subtypes, epitopes within the HA head—specifically at antigenic sites Sa, Sb, Ca1, Ca2, and Cb—are known to elicit subtype-specific immune responses. The HA stalk region, by contrast, contains more broadly conserved epitopes that can induce cross-reactive immunity across subtypes. Similarly, in the H3N2 subtype, key epitopes are in antigenic sites A, B, C, D, and E, resembling those in human H3N2 subtypes, with its stalk region also contributing to broadly protective immune responses.

The neuraminidase (NA) protein is another critical antigenic target, particularly for anti-neuraminidase antibodies that help limit viral spread. In the N1 subtype (as seen in trH1N1 and trH1N2), epitopes are present in the enzymatic active site, while the N2 subtype (trH3N2) contains epitopes homologous to those found in seasonal human H3N2 viruses. NA stalk domains also harbor conserved epitopes contributing to cross-reactive immunity across diverse influenza subtypes.

In addition to antibody-mediated responses, T-cell immunity plays a pivotal role in controlling AIV infections by recognizing conserved internal viral protein epitopes. The nucleoprotein (NP) contains well-characterized CD8+ T-cell epitopes, including NP₃₃₈₋₃₄₅ (IYSTVASSL) and NP₃₆₆₋₃₇₄ (ASNENMETM), which are conserved across influenza A subtypes. Matrix protein 1 (M1) also presents a dominant cytotoxic T lymphocyte (CTL) epitope, MI₅₈₋₆₆ (GILGFVFTL), commonly used in vaccine design. Furthermore, polymerase proteins PA, PB1, and PB2 possess additional conserved CD8+ T-cell epitopes, further enhancing the cellular immune response against diverse AIV subtypes.

The identified epitope sequences are based on consensus regions observed across multiple studies. However, variations may exist among influenza subtypes, and precise epitope mapping should consider specific viral sequences. Understanding these conserved epitopes provides valuable insights for vaccine design and antiviral strategies against AIV (Table 1).

A recombinant process can produce the antigen or RNA, including a linear mRNA, a circular RNA, and both with and without self-replication.

A recombinant process can produce the AIV, cell-free synthesis using mRNA or directly injecting mRNA capable of expressing said antigens. The mRNA can be linear or circular and has self-replicating properties besides single-cycle expression.

The relative quantitative yield of protein expression from the four RNA types can be approximated based on experimental data and known differences in stability, replication, and translation efficiency. While absolute yields depend on experimental conditions, approximate yield ratios can range from 100× to 1,000,000×.

In one embodiment, the present invention can be designed with any peptide polypeptide or protein as carriers fused to various epitopes derived from AIV.

In one embodiment, the universal vaccine of the present invention could be administered by syringe injection, needle-free injection, microneedle patch, and delivery. The pharmaceutical combination can be administered by different routes, such as oral, intramuscular (IM), subcutaneous (SC), intradermal (ID), and intranasal spray (INS).

In one embodiment, the present invention is suitable to qualify as an emergency vaccine under the OIE protocol to be used against outbreaks of emerging AIV subtypes. This qualification is achieved because this vaccine provides birds with sufficient protection against AIV infection after a single administration.

In one embodiment, the present invention is suitable for generating antigen banks that could be used in an emergency to formulate an AIV universal vaccine.

In another embodiment, the vaccine formulations can be administered in multiple doses.

In one embodiment, the present invention provides a vaccine formulation capable of inducing cross-protection against different subtypes of AIV, comprising AIV peptides, polypeptides, or proteins coded in plasmids; b) recombinant AIV peptides, polypeptides or proteins; c) peptides, polypeptides or proteins used as a carrier or as molecular adjuvant fused to peptides, polypeptides and/or proteins derived from AIV; d) adjuvants; emulsifiers, molecular adjuvants and carrier systems.

In one embodiment, the above vaccine formulation can induce protective immunity against all subtypes of AIV. In another embodiment, the vaccine formulation can induce protective immunity against all subtypes of one or more subtypes. In another embodiment, the vaccine formulation can induce protective immunity against all subtypes of AIV.

The vaccine formulation comprises a lipid nanoparticle (LNP) in one embodiment.

In one embodiment, the carrier systems can be liposomes, microspheres, nanoparticles, micellar systems, or immune stimulating complexes (ISCOMs).

The present invention also provides a method of vaccinating a host susceptible to AIV infection, comprising administering the vaccine formulation described above to the host to induce an immune response.

In one embodiment, the vaccine formulation can induce protective immunity against all subtypes of a given virus or against all known subtypes of said virus.

The present invention also provides a method of vaccinating a host susceptible to AIV infection, comprising administering the vaccine formulation described above to the host to induce an immune response. In some embodiments, the vaccine formulation components are administrated simultaneously but in different locations of the host. In certain embodiments, the components of the vaccine formulation are administrated at different time points in the same location as the host. In one embodiment, the components of the vaccine formulation are administrated at different time points in other places of the host.

The host has not been infected with AIV in one embodiment, and the induced immune response is protective. In another embodiment, the induced immune response is the humoral or cellular immune response.

In one embodiment, the induced immune response comprises cross-protective neutralizing antibodies against various subtypes of AIV. In another embodiment, the induced immune response cross-reacts against various subtypes of AIV.

In some embodiments, the vaccine formulation used in the pharmaceutical combination of the present invention can induce cross-protection against different subtypes of a target virus, AIV.

In one embodiment, the carrier protein-protein is fused to the components that can trigger immune responses by peptide or polypeptide linkers.

In one embodiment, the pharmaceutical combination can be administered simultaneously, along with the exact body location of the host. In another embodiment, the pharmaceutical combination can be administered at different times and the precise body location of the host. In another embodiment, the pharmaceutical combination can be administered simultaneously and at various body locations of the host. In another embodiment, the pharmaceutical combination can be administered at different times and other body locations of the host.

1. A universal avian influenza vaccine formulation designed to confer cross-protection against multiple AIV subtypes, comprising a plurality of conserved peptide epitopes derived from the hemagglutinin and neuraminidase proteins encoded by H1N1, H1N2, and H3N2 subtypes.

2. The universal avian influenza vaccine formulation of claim 1, wherein the peptide epitopes comprise PQRERRRKK (SEQUENCE NO. 1), LKNNQKIYVQTL (SEQUENCE NO. 2), WLTEKEGSYP (SEQUENCE NO. 3), GLFGAIAGFIE (SEQUENCE NO. 4), VPNLPFQNAL (SEQUENCE NO. 5), RTFFLTQGA (SEQUENCE NO. 6), ILRTQESEC (SEQUENCE NO. 7), IASRSGYSGN (SEQUENCE NO. 8), PCICITPNGSIP (SEQUENCE NO. 9), KWVDGTQYRT (SEQUENCE NO. 10), CTELKLSDY (SEQUENCE NO. 11), RRSGAAGAAVK (SEQUENCE NO. 12), ILRGSVAHK (SEQUENCE NO. 13), ELRSRYWAI (SEQUENCE NO. 14), GILGFVFTL (SEQUENCE NO. 15), ASCMGLIY (SEQUENCE NO. 16), SLLTEVETPIRNEWGCRCNDSSD (SEQUENCE NO. 17), SLLTEVETPTRSEWECRCSDSSD (SEQUENCE NO. 18), and SLLTEVETPTRNGWECKCSDSSD (SEQUENCE NO. 19), or a combination thereof.

3. The universal avian influenza vaccine formulation of claim 1, wherein the peptide epitopes comprise SLLTEVETPIRNEWGCRCNDSSD (SEQUENCE NO. 20), GLFGAIAGFIE (SEQUENCE NO. 21), ELRSRYWAI (SEQUENCE NO. 22), GILGFVFTL (SEQUENCE NO. 23) and ILRTQESEC (SEQUENCE NO. 24), or a combination thereof.

4. The universal avian influenza vaccine formulation of claim 1, wherein the linkers comprise RVRRKR (SEQUENCE NO. 25), a Furin cleavage site between the hemagglutinin conserved region and other epitopes, EAAAKEAAA (SEQUENCE NO. 26), a rigid helical linker to maintain spatial separation for the fusion peptide, and SLLRY (SEQUENCE NO. 27), a cathepsin linker, or a combination thereof.

5. The universal avian influenza vaccine formulation of claim 1, wherein the preferred sequence of peptide for recombinant expression comprises SLLTEVETPIRNEWGCRCNDSSD-RVRRKR-GLFGAIAGFIE-EAAAKEAAAK-ELRSRYWAI-SLLRY-GILGFVFTL-SLLRY-ILRTQESEC (SEQUENCE NO. 28).

6. The universal avian influenza vaccine formulation of claim 1, wherein an open reading frame for an RNA expression comprises SLLTEVETPIRNEWGCRCNDSSD-GGGGS-GLFGAIAGFIE-AAY-ELRSRYWAI-GPGPG-GILGFVFTL-GPGPG-ILRTQESEC (SEQUENCE NO. 29).

7. The universal avian influenza vaccine formulation of claim 1, wherein the peptide epitopes are joined by a flexible peptide linker or a cleavable linker.

8. The universal avian influenza vaccine formulation of claim 1, wherein the vaccine is formulated using biodegradable nanoparticles or microparticles, made from poly(lactic-co-glycolic acid) (PLGA) or chitosan.

9. The universal avian influenza vaccine formulation of claim 1, wherein the vaccine is formulated by encapsulating antigens in liposomes or immunostimulating complexes (ISCOMs).

10. The universal avian influenza vaccine formulation of claim 1, wherein the vaccine is formulated using adjuvants such as Montanide ISA 70 or ISA 71.

11. The universal avian influenza vaccine formulation of claim 1, wherein the vaccine is produced by a recombinant or RNA-based process, either through cell-free synthesis or by in vivo expression following administration.

12. The universal avian influenza vaccine formulation of claim 1, wherein peptide epitopes are derived from the same or different AIV strain(s) or subtypes(s).

13. A method of vaccinating a host susceptible to AIV infection, comprising administering one or more of the vaccine formulations of claim 1 to induce an immune response in a host comprising an avian species.

14. The method of vaccinating of claim 13, wherein the induced immune response is a humoral or cellular immune response.

15. The method of claim 13, wherein the induced immune response comprises cross-protective neutralizing antibodies against two or more subtypes of AIV.

16. The method of vaccinating of claim 9, wherein an intramuscular, subcutaneous, intravenous, or intranasal route administers the vaccine.


SEQUENCE LISTING (29 sequences)

SEQ ID NO: 1 - protein, 9 aa, Alphainfluenzavirus influenzae
PQRERRRKK

SEQ ID NO: 2 - protein, 12 aa, Alphainfluenzavirus influenzae
LKNNQKIYVQTL

SEQ ID NO: 3 - protein, 10 aa, Alphainfluenzavirus influenzae
WLTEKEGSYP

SEQ ID NO: 4 - protein, 11 aa, Alphainfluenzavirus influenzae
GLFGAIAGFIE

SEQ ID NO: 5 - protein, 11 aa, Alphainfluenzavirus influenzae
GLFGAIAGFIE

SEQ ID NO: 6 - protein, 9 aa, Alphainfluenzavirus influenzae
RTFFLTQGA

SEQ ID NO: 7 - protein, 9 aa, Alphainfluenzavirus influenzae
ILRTQESEC

SEQ ID NO: 8 - protein, 10 aa, Alphainfluenzavirus influenzae
IASRSGYSGN

SEQ ID NO: 9 - protein, 12 aa, Alphainfluenzavirus influenzae
PCICITPNGSIP

SEQ ID NO: 10 - protein, 10 aa, Alphainfluenzavirus influenzae
KWVDGTQYRT

SEQ ID NO: 11 - protein, 9 aa, Alphainfluenzavirus influenzae
CTELKLSDY

SEQ ID NO: 12 - protein, 11 aa, Alphainfluenzavirus influenzae
RRSGAAGAAVK

SEQ ID NO: 13 - protein, 9 aa, Alphainfluenzavirus influenzae
ILRGSVAHK

SEQ ID NO: 14 - protein, 9 aa, Alphainfluenzavirus influenzae
ELRSRYWAI

SEQ ID NO: 15 - protein, 9 aa, Alphainfluenzavirus influenzae
GILGFVFTL

SEQ ID NO: 16 - protein, 8 aa, Alphainfluenzavirus influenzae
ASCMGLIY

SEQ ID NO: 17 - protein, 23 aa, Alphainfluenzavirus influenzae
SLLTEVETPIRNEWGCRCNDSSD

SEQ ID NO: 18 - protein, 23 aa, Alphainfluenzavirus influenzae
SLLTEVETPTRSEWECRCSDSSD

SEQ ID NO: 19 - protein, 23 aa, Alphainfluenzavirus influenzae
SLLTEVETPTRNGWECKCSDSSD

SEQ ID NO: 20 - protein, 23 aa, Alphainfluenzavirus influenzae
SLLTEVETPIRNEWGCRCNDSSD

SEQ ID NO: 21 - protein, 11 aa, Alphainfluenzavirus influenzae
GLFGAIAGFIE

SEQ ID NO: 22 - protein, 9 aa, Alphainfluenzavirus influenzae
ELRSRYWAI

SEQ ID NO: 23 - protein, 9 aa, Alphainfluenzavirus influenzae
GILGFVFTL

SEQ ID NO: 24 - protein, 9 aa, Alphainfluenzavirus influenzae
ILRTQESEC

SEQ ID NO: 25 - protein, 6 aa, Synthetic construct
RVRRKR

SEQ ID NO: 26 - protein, 9 aa, Synthetic construct
EAAAKEAAA

SEQ ID NO: 27 - protein, 5 aa, Synthetic construct
SLLRY

SEQ ID NO: 28 - protein, 87 aa, Synthetic construct
SLLTEVETPIRNEWGCRCNDSSDRVRRKRGLFGAIAGFIEEAAAKEAAAKELRSRYWAISLLRYGILGFVFTLSLLRYILRTQESEC

SEQ ID NO: 29 - protein, 79 aa, Synthetic construct
SLLTEVETPIRNEWGCRCNDSSDGGGGSGLFGAIAGFIEAAYELRSRYWAIGPGPGGILGFVFTLGPGPGILRTQESEC